(19) United States
(12) Patent Application Publication
(10) Pub. No.: US 2013/0006317 A1
(43) Pub. Date: Jan. 3, 2013

(54) DEVICES, SYSTEMS AND METHODS TO ANALYZE EVOKED RESPONSES TO PRE-PACING PULSES TO PREDICT IMMINENT VT/VF, ESTIMATE ISCHEMIC BURDEN AND/OR CHARACTERIZE ELECTRICAL SUBSTRATES

(76) Inventors: Allen J. Keel, San Francisco, CA (US); Kyungmoo Ryu, Palmdale, CA (US); Stuart Rosenberg, Castaic, CA (US); Kiell Norén, Solna (SE)

(21) Appl. No.: 13/174,307

(22) Filed: Jun. 30, 2011

(51) Int. Cl. A61N 1/368; A61N 1/39; A61N 1/362

(52) U.S. Cl. 607/4; 607/27; 607/17

(57) ABSTRACT

Described herein are implantable systems, and methods for use therewith, to predict whether ventricular tachycardia (VT) or ventricular fibrillation (VF) is imminent, estimate ischemic burden and/or characterize an electrical substrate of the LV chamber. For each of a plurality of cardiac cycles, a pacing vector comprising a first set of electrodes is used to deliver a pre-pacing pulse at a site within the LV chamber (wherein the pre-pacing pulse is delivered prior to an intrinsic activation of the LV chamber), and a sensing vector comprising a second set of electrodes is used to detect an evoked response to the pre-pacing pulse. The detected evoked responses to the pre-pacing pulses are analyzed, and results of the analysis are used predict whether VT or VF is imminent, estimate ischemic burden and/or characterize an electrical substrate of the LV chamber.

### FIELD OF THE INVENTION

Embodiments of the present invention generally relate to implantable cardiac stimulation devices, and methods for use therewith, that can be used to predict imminent ventricular tachycardia (VT) or ventricular fibrillation (VF), estimate ischemic burden and/or characterize electrical substrates of the heart.

### BACKGROUND

Ventricular arrhythmias, which are arrhythmias that originate in the ventricles, include ventricular tachycardia (VT) and ventricular fibrillation (VF). Ventricular arrhythmias are often associated with rapid and/or chaotic ventricular rhythms. VT episodes have the potential to suddenly develop into a faster, polymorphic version that is life-threatening, the worst case being an end result of a virtually hemodynamicless VF. In VF, disorganized action potentials can cause the myocardium to quiver rather than contract. Such chaotic quivering can greatly reduce the heart's pumping ability. Once in VF, a human's physical state is severely compromised, and VF must be converted to sinus rhythm within a very short amount of time to prevent mortality or irreversible brain damage. Indeed, approximately two-thirds of all deaths from arrhythmia are caused by VF. A variety of conditions such as, but not limited to, hypoxia, ischemia, pharmacologic therapy (e.g., sympathomimetics), and asynchronous pacing may promote onset of ventricular arrhythmia.

VF is typically fatal if not terminated within minutes using shock therapy. VT can be lethal if not treated promptly, and is usually treated using either anti-tachycardia pacing (ATP) or shock therapy to terminate an episode of VT.

The advent of implantable cardio-defibrillators (ICDs) allows for VT and VF to be detected in real-time and for therapy (shock or anti-tachycardia pacing) to be delivered accordingly. However, due to the time sensitivity of the adverse effects of VT and VF, it would be advantageous to predict the imminent onset of VT and VF, with the aim of either preventing VT and VF, preventing VT from progressing into VF, or shortening the duration of VT or VF.

Ischemic burden refers to the total amount of both asymptomatic and symptomatic episodes of myocardial ischemia. Myocardial ischemia, which involves oxygen starvation of the myocardium, can lead to myocardial infarction and/or the onset of malignant arrhythmias if the oxygen starvation is not alleviated. Although myocardial ischemia is sometimes associated with the symptom of angina pectoris (i.e., chest pain), the majority of episodes of myocardial ischemia are asymptomatic or “silent.”

A wide range of therapies are known for the treatment of myocardial ischemia once it is detected, including surgical revascularization, neural stimulation and use of a variety of biologically active agents or compounds which can remove blood clots, reduce cardiac workload or improve cardiac circulation. However, accurate and rapid detection of myocardial ischemia is necessary in order to reduce the morbidity and mortality from this often silent but deadly condition. In other words, without knowledge of the condition, it cannot be treated.

A higher total ischemic burden has been shown to be correlated with a greater chance of future adverse coronary events and greater morbidity and mortality in patients. Zones of ischemia can create lines of structural or functional block, which slow down electrical conduction, leading to sub-optimal dyssynchronous mechanical activation and reduced pump efficiency of the heart.

Accordingly, it would also be advantageous to provide new and improved techniques and systems for monitoring myocardial ischemic burden.

### SUMMARY

Embodiments of the present invention relate to implantable systems, and methods for use therewith, to predict whether ventricular tachycardia (VT) or ventricular fibrillation (VF) is imminent, estimate ischemic burden and/or characterize an electrical substrate of the LV chamber. For each of a plurality of cardiac cycles, a pacing vector comprising a first set of electrodes is used to deliver a pre-pacing pulse at a site within the LV chamber (wherein the pre-pacing pulse is delivered prior to an intrinsic activation of the LV chamber), and a sensing vector comprising a second set of electrodes is used to detect an evoked response to the pre-pacing pulse. The detected evoked responses to the pre-pacing pulses are analyzed, and results of the analysis are used predict whether VT or VF is imminent, estimate ischemic burden and/or characterize an electrical substrate of the LV chamber.

A pre-pacing pulse can be delivered to a single site within the LV chamber. Alternatively, a pre-pacing pulse can be delivered to multiple sites within the LV chamber. Where a pre-pacing pulse is delivered to multiple sites, multiple pacing vectors are used. Multisite pre-pacing may be used, e.g., if single-site pre-pacing yields a sensed evoked response that is insufficient for waveform analysis. Further, as explained below, in certain embodiments a pre-pacing pulse can be delivered to a single site within the LV chamber for a plurality of cardiac cycles, and to multiple sites within the LV chamber for a further plurality of cardiac cycles, thereby enabling the evoked responses to single site pre-pacing to be compared to evoked responses to multi-site pre-pacing.

In accordance with specific embodiments, the evoked responses to the pre-pacing pulses can be analyzed by determining one or more predetermined features of each of the evoked responses to the pre-pacing pulses, and determining a measure of variation for each of the one or more predetermined features. The one or more measures of variation can then be used to predict whether VT or VF is imminent, estimate ischemic burden and/or characterize an electrical substrate of the LV chamber.

The one or more predetermined features of each of the evoked responses can include one or more features of an R-wave indicative of ventricular depolarization and/or one or more features of a T-wave indicative of ventricular repolarization. For example, these features can include one or more of: maximum upward slope of the R-wave, maximum amplitude of the R-wave, maximum downward slope of the R-wave, maximum negative dV/dt of the R-wave, time from delivery of the pre-pacing pulse to a predetermined feature of the R-wave, number of deflections of the R-wave, number of peaks of the R-wave, integral of the R-wave, Fast Fourier Transform (FFT) features, maximum amplitude of the T-wave, integral of the T-wave, time from delivery of the pre-pacing pulse to onset of the T-wave, and time from delivery of the pre-pacing pulse to peak of the T-wave, but are not limited thereto.

Exemplary measures of variation, which can be determined for one or more features of the evoked responses, can include one or more of: standard deviation, normalized standard deviation, interquartile range, range, mean difference, median absolute deviation, average absolute deviation, coefficient of variation, quartile coefficient of dispersion, relative mean difference, variance, and variance-to-mean ratio of the metric, but are not limited thereto.

In accordance with an embodiment, the one or more measures of variation is/are compared to one or more VT variation threshold(s) that if exceeded is/are indicative of VT being imminent. Additionally, the one or more measures of variation is/are compared to one or more VF variation threshold(s) that if exceeded is/are indicative of VF being imminent (wherein each VF variation threshold is greater than a corresponding VT variation threshold). A determination of whether VT or VF is imminent is based on the results of the aforementioned comparisons. In response to predicting that VT is imminent, ventricular overdrive pacing can be delivered to reduce a chance of VT sustaining or accelerating into a fast polymorphic VT or VF. In response to predicting that VF is imminent, one or more capacitors used for delivering a defibrillation shock can begin to be charged, so that a shock can be delivered more quickly once VF is detected.

In accordance with specific embodiments, the evoked responses to the pre-pacing pulses can be analyzed by determining an extent of beat-to-beat alternans associated with the evoked responses. The extent of beat-to-beat alternans can then be used to predict whether VT or VF is imminent, estimate ischemic burden and/or characterize an electrical substrate of the LV chamber.

In accordance with specific embodiments, evoked responses to the pre-pacing pulses can be analyzed by determining an extent of fractionation of portions of an electrocardiogram indicative of the evoked responses. The extent of the fractionation can then be used to estimate ischemic burden and/or characterizing an electrical substrate of the LV chamber.

In accordance with alternative embodiments, the evoked responses to the pre-pacing pulses can be analyzed by determining a conduction time delay from each pre-pacing pulse to a predetermined feature of an R-wave or T-wave resulting from the pre-pacing pulse. In such embodiments, an ischemic burden can be estimated and/or an electrical substrate of the LV chamber can be characterized based on lengths of the conduction time delays. More specifically, it is believed that an increase in the conduction time delays is indicative increased ischemic burden, as well as increased functional and/or structural block of an electrical substrate of the LV chamber.

As mentioned above, the pre-pacing pulses can be delivered at a single site within the LV chamber, or at multiple sites within the LV chamber. In certain embodiments, pre-pacing pulses are delivered at a single site for a plurality of cardiac cycles, and at two sites for a further plurality of cardiac cycles. In such embodiments, as part of the analysis of the evoked responses, evoked responses to the two site pre-pacing pulses can be compared to the evoked responses to the single site pre-pacing pulses to estimate ischemic burden and/or characterize an electrical substrate of the LV chamber. This can include using results of the comparisons to estimate a location of an ischemic region within the LV chamber.

In accordance with specific embodiments of the present invention where ischemic burden is estimated, the estimated ischemic burden can be used to selecting how many sites within the LV chamber is/are to be paced as part of cardiac resynchronization therapy (CRT).

This summary is not intended to be a complete description of, or limit the scope of, the invention. Alternative and additional features, aspects, and objects of the invention can be obtained from a review of the specification, the figures, and the claims.

### BRIEF DESCRIPTION OF THE DRAWINGS

FIG. 1A is a simplified, partly cutaway view illustrating an implantable stimulation device in electrical communication with at least three leads implanted into a patient's heart for delivering multi-chamber stimulation and shock therapy and sensing cardiac activity.

FIG. 1B is a functional block diagram of the multi-chamber implantable stimulation device of FIG. 1A, illustrating the basic elements that provide pacing stimulation, cardioversion, and defibrillation in four chambers of the heart.

FIG. 2 is a high level flow diagram that is used to describe techniques to predict whether VT or VF is imminent, according to embodiments of the present invention.

FIG. 3 illustrates an exemplary evoked response to a pre-pacing pulse and exemplary features of the evoked response.

FIG. 4 is a high level flow diagram that is used to describe techniques to estimate a patients ischemic burden, according to embodiments of the present invention.

FIG. 5 illustrates morphologies of evoked responses to pre-pacing pulses where a subject has no myocardial ischemia, as well as where a subject has myocardial ischemia.

FIG. 6 illustrates beat-to-beat morphology differences for evoked responses to pre-pacing pulses where a subject has no myocardial ischemia, as well as where a subject has myocardial ischemia.

FIGS. 7A and 7B are used to illustrate how MSLV pacing can create flat wavefronts that can break through functional block caused by ischemic zones.

FIG. 8 is a high level flow diagram that is used to describe techniques to characterize an electrical substrate of a patient's LV chamber, according to embodiments of the present invention.

### DETAILED DESCRIPTION OF THE PREFERRED EMBODIMENTS

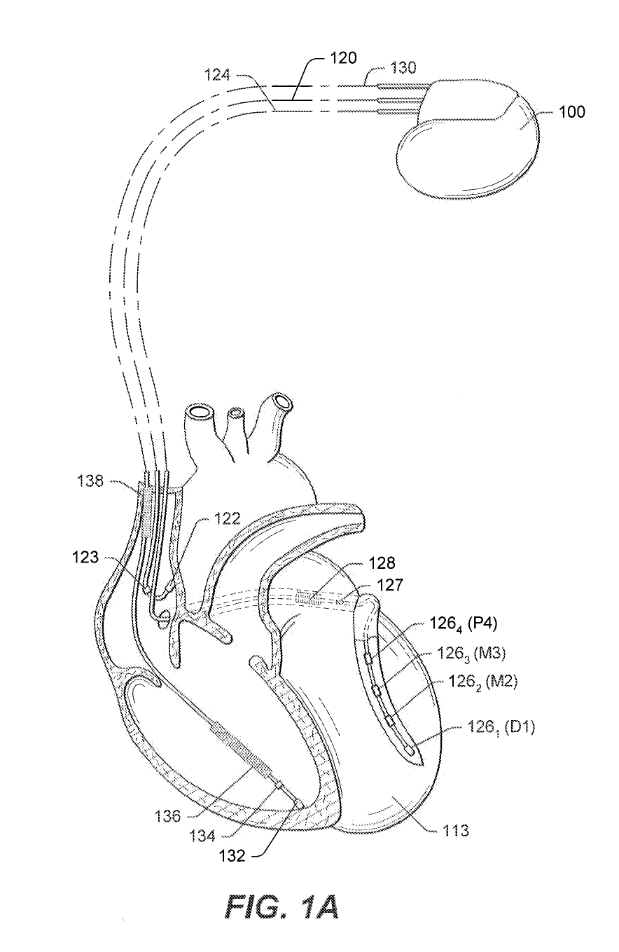

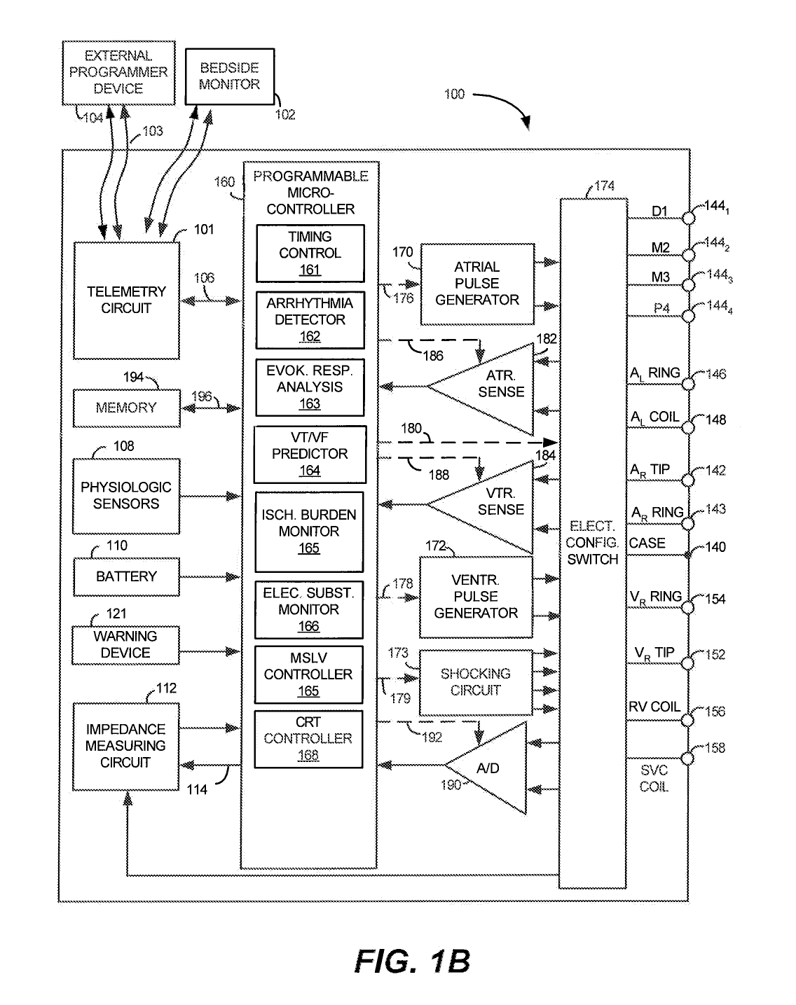

Embodiments of the present invention generally relate to chronically implantable cardiac stimulation devices and systems such as pacemakers and/or implantable cardioverter-defibrillators (ICDs) and methods for use therewith. In particular, embodiments of the present invention can be used to predict whether ventricular tachycardia (VT) or ventricular fibrillation (VF) is imminent, estimate a patient's ischemic burden and/or characterize an electrical substrate of a patient's LV chamber. While not limited thereto, such embodiments are especially useful with implantable devices and systems capable of multi-site left ventricular (MSLV) pacing. In view of the above, an exemplary implantable cardiac system capable of delivering MSLV pacing, in which embodiments of the present invention described herein could be implemented, will now be described in conjunction with FIGS. 1A and 1B.

### Exemplary Pacemaker/ICD

With reference to FIGS. 1A and 1B, a description of an exemplary pacemaker/ICD will now be provided. FIG. 1A provides a simplified block diagram of the pacemaker/ICD, which is a dual-chamber stimulation device 100 capable of treating both fast and slow arrhythmias with stimulation therapy, including cardioversion, defibrillation, and pacing stimulation, including MSLV pacing. To provide atrial chamber pacing stimulation and sensing, pacemaker/ICD 100 is shown in electrical communication with a heart 113 by way of a right atrial (RA) lead 120 having an atrial tip electrode 122 and an atrial ring electrode 123 implanted in the atrial appendage. Pacemaker/ICD 100 is also in electrical communication with the heart by way of a right ventricular (RV) lead 130 having, in this embodiment, a ventricular tip electrode 132, a RV ring electrode 134, a RV coil electrode 136, and a superior vena cava (SVC) coil electrode 138. Typically, the RV lead 130 is transvenously inserted into the heart so as to place the RV coil electrode 136 in the RV apex, and the SVC coil electrode 138 in the superior vena cava. Accordingly, the RV lead is capable of receiving cardiac signals, and delivering stimulation in the form of pacing and shock therapy to the right ventricle (also referred to as the RV chamber).

To sense left atrial and ventricular cardiac signals and to provide left chamber pacing therapy, pacemaker/ICD 100 is coupled to a multi-pole LV lead 124 designed for placement in the “CS region” via the CS os for positioning a distal electrode adjacent to the left ventricle and/or additional electrode(s) adjacent to the left atrium (also referred to as the LA chamber). As used herein, the phrase “CS region” refers to the venous vasculature of the left ventricle, including any portion of the CS, great cardiac vein, left marginal vein, left posterior ventricular vein, middle cardiac vein, and/or small cardiac vein or any other cardiac vein accessible by the CS. Accordingly, an exemplary LV lead 124 is designed to receive atrial and ventricular cardiac signals and to deliver left ventricular pacing therapy using a set of four LV electrodes 126₁, 126₂, 126₃, and 126₄ (thereby providing a quadra-pole lead), left atrial pacing therapy using at least a LA ring electrode 127, and shocking therapy using at least a LA coil electrode 128. In certain embodiments, the LV lead 124 includes the LV electrodes 126₁, 126₂, 126₃, and 126₄, but does not include the LA electrodes 127 and 128. Such a lead can be, e.g., the Quartet™ left ventricular pacing lead developed by St. Jude Medical Inc. (headquartered in St. Paul, Minn.), which includes four pacing electrodes on the left ventricular lead—enabling up to 10 pacing configurations.

The LV electrode 126₁ is shown as being the most “distal” LV electrode (with relation to how far the electrode is from where the LV lead 124 connects to the pacemaker/ICD 100). The LV electrode 126₄ is shown as being the most “proximal” LV electrode. The LV electrodes 126₂ and 126₃ are shown as being “middle” LV electrodes, between the distal and proximal LV electrodes 126₁ and 126₄. Accordingly, so as to more aptly describe their relative locations, the four LV electrodes 126₁, 126₂, 126₃, and 126₄ can be referred to respectively as electrodes D1, M2, M3 and P4 (where “D” stands for “distal”, “M” stands for “middle”, and “P” stands from “proximal”, and the numbers are arranged from most distal to most proximal).

It is also possible that more or fewer LV electrodes are provided. However, for much of the remaining discussion, it will be assumed that the multi-pole LV lead 124 includes the four LV electrodes 126₁, 126₂, 126₃, and 126₄ (i.e., LV electrodes D1, M2, M3 and P4, respectively).

The four LV electrodes can be used to provide various different pacing vectors and sensing vectors. Some of the vectors are intraventricular LV vectors (vectors between two LV electrodes); whereas others are interventricular vectors (e.g., vectors between a LV electrode and the RV coil 136). Below is a list of exemplary vectors that can be used for pacing and/or sensing using the LV electrodes D1, M2, M3 and P4 with and without the RV coil 136. In the following list, the first electrode in each row (i.e., the electrode to the left of the arrow) is assumed to be connected as the cathode, and the second electrode in each row (i.e., the electrode to the right of the arrow) is assumed to be connected as the anode, but that need not be the case, especially where neither electrode is a coil.

D1→RV coil

M2→RV coil

M3→RV coil

P4→RV coil

D1→M2

D1→P4

M2→P4

M3→M2

M3→P4

P4→M2

Alternative and/or additional vectors, other than those listed above, can be used for pacing and/or sensing. Although only three leads are shown in FIG. 1A, it should also be understood that additional leads (with one or more pacing, sensing and/or shocking electrodes) might be used and/or additional electrodes might be provided on the leads already shown, such as additional electrodes on the RV or LV lead. It is also possible that less than three leads be used.

A simplified block diagram of internal components of pacemaker/ICD 100 is shown in FIG. 18. While a particular pacemaker/ICD is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation. The housing 140 for pacemaker/ICD 100, shown schematically in FIG. 1B, is often referred to as the “can”, “case” or “case electrode” and may be programrnably selected to act as the return electrode for all “unipolar” modes. The housing 140 may further be used as a return electrode alone or in combination with one or more of the coil electrodes, 128, 136 and 138, for shocking purposes. The housing 140 further includes a connector (not shown) having a plurality of terminals, 142, 143, 144₁-144₄, 146, 148, 152, 154, 156 and 158 (shown schematically and, for convenience, the names of the electrodes to which they are connected are shown next to the terminals). As such, to achieve RA sensing and pacing, the connector includes at least a RA tip terminal (A_{R} TIP) 142 adapted for connection to the atrial tip electrode 122 and a RA ring (A_{R} RING) electrode 143 adapted for connection to RA ring electrode 123. To achieve left chamber sensing, pacing and shocking, the connector includes a LV tip terminal 144₁ adapted for connection to the D1 electrode and additional LV electrode terminals 144₂, 144₃ and 144₄ terminals adapted for connection to the M2, M3 and P4 electrodes of the quadra-pole LV lead.

The connector also includes a LA ring terminal (A_{L} RING) 146 and a LA shocking terminal (A_{L} COIL) 148, which are adapted for connection to the LA ring electrode 127 and the LA coil (A_{L} COIL) electrode 128, respectively. To support right chamber sensing, pacing and shocking, the connector further includes a RV tip terminal (V_{R} TIP) 142, a RV ring terminal (V_{R} RING) 143, a RV shocking terminal (V_{R} COIL) 156, and an SVC shocking terminal (SVC COIL) 158, which are adapted for connection to the RV tip electrode 132, RV ring electrode 134, the RV coil electrode 136, and the SVC coil electrode 138, respectively.

At the core of pacemaker/ICD 100 is a programmable microcontroller 160, which controls the various modes of stimulation therapy. As is well known in the art, the microcontroller 160 (also referred to herein as a control unit or controller) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 160 includes the ability to process or monitor input signals (data) as controlled by a program code stored in a designated block of memory. The details of the design and operation of the microcontroller 160 are not critical to the invention. Rather, any suitable microcontroller 160 may be used that carries out the functions described herein. The use of microprocessor-based control circuits for performing timing and data analysis functions are well known in the art.

As shown in FIG. 1B, an atrial pulse generator 170 and a ventricular pulse generator 172 generate pacing stimulation pulses for delivery by the RA lead 120, the RV lead 130, and/or the LV lead 124 via an electrode configuration switch 174. It is understood that in order to provide stimulation therapy in each of the four chambers of the heart, the atrial and ventricular pulse generators, 170 and 172, may include dedicated, independent pulse generators, multiplexed pulse generators or shared pulse generators. The pulse generators, 170 and 172, are controlled by the microcontroller 160 via appropriate control signals, 176 and 178, respectively, to trigger or inhibit the stimulation pulses. The microcontroller 160 includes timing control circuitry 161 to control the timing of the stimulation pulses, including, but not limited to, pacing rate, atrio-ventricular (AV) delay, interatrial conduction (AA) delay, interventricular conduction (VV) delay and/or intraventricular delay (e.g., LV1-LV2 delay). The timing control circuitry 161 can also keep track of the timing of refractory periods, blanking intervals, noise detection windows, evoked response detection windows, alert intervals, marker channel timing, etc., which is well known in the art.

The microcontroller 160 further includes an arrhythmia detector 162. The detector 162 can be utilized by the stimulation device 100 for determining desirable times to administer various therapies. The detector 162 may be implemented in hardware as part of the microcontroller 160, or as software/firmware instructions programmed into the device and executed on the microcontroller 160 during certain modes of operation.

The microcontroller 160 further includes an evoked response analysis module 163, a VT/VF predictor module 164, an ischemic burden monitor module 165 and an electrical substrate monitor module 166. These modules can be used to implement various exemplary algorithms and/or methods presented below. The aforementioned components may be implemented in hardware as part of the microcontroller 260, or as software/firmware instructions programmed into the device and executed on the microcontroller 160 during certain modes of operation. The evoked response analysis module 163, as described herein, may aid in the acquisition, analysis, etc., of information related to IEGMs and, in particular, analyzing evoked responses to pre-pacing pulses delivered in accordance with embodiments of the present invention. The VT/VF predictor module 164 may aid in the prediction of whether VT or VF is imminent. The ischemic burden monitor module 165 may aid in monitoring a patient's ischemic burden. The electrical substrate monitor module 166 may aid in the monitoring of an electrical substrate of a patient's LV chamber.

Additional components of the microcontroller include a MSLV controller 167 to control the actual delivery of MSLV pacing and a CRT controller 168 to control CRT, which can be performed in conjunction with MSLV pacing.

Depending upon the implementation, the various components of the microcontroller may be implemented as separate software modules or the modules may be combined to permit a single module to perform multiple functions. For example, the MSLV controller and the CRT controller 168 can be combined. In addition, although shown as being components of the microcontroller, some or all of these components may be implemented separately from the microcontroller, using application specific integrated circuits (ASICs) or the like.

Switch 174 includes a plurality of switches for connecting the desired electrodes to the appropriate I/O circuits, thereby providing complete electrode programmability. Accordingly, the switch 174, in response to a control signal 180 from the microcontroller 160, determines the polarity of the stimulation pulses (e.g., unipolar, bipolar, combipolar, etc.) by selectively closing the appropriate combination of switches (not shown) as is known in the art. The switch also switches among the various LV electrodes.

Atrial sensing circuits 182 and ventricular sensing circuits 184 may also be selectively coupled to the RA lead 120, LV lead 124, and the RV lead 130, through the switch 174 for detecting the presence of cardiac activity in each of the four chambers of the heart. Accordingly, the atrial (ATR. SENSE) and ventricular (VTR. SENSE) sensing circuits, 182 and 184, may include dedicated sense amplifiers, multiplexed amplifiers or shared amplifiers. The switch 174 determines the “sensing polarity” of the cardiac signal by selectively closing the appropriate switches, as is also known in the art. In this way, the clinician may program the sensing polarity independent of the stimulation polarity. Each sensing circuit, 182 and 184, preferably employs one or more low power, precision amplifiers with programmable gain and/or automatic gain control, bandpass filtering, and a threshold detection circuit, as known in the art, to selectively sense the cardiac signal of interest. The automatic gain control enables pacemaker/ICD 100 to deal effectively with the difficult problem of sensing the low amplitude signal characteristics of atrial or ventricular fibrillation. The outputs of the atrial and ventricular sensing circuits, 182 and 184, are connected to the microcontroller 160 which, in turn, are able to trigger or inhibit the atrial and ventricular pulse generators, 170 and 172, respectively, in a demand fashion in response to the absence or presence of cardiac activity in the appropriate chambers of the heart.

For arrhythmia detection, pacemaker/ICD 100 utilizes the atrial and ventricular sensing circuits, 182 and 184, to sense cardiac signals to determine whether a rhythm is physiologic or pathologic. As used in this section “sensing” is reserved for the noting of an electrical signal, and “detection” is the processing of these sensed signals and noting the presence of an arrhythmia, an evoked response, an intrinsic event, or some other event being monitored for. The timing intervals between sensed events (e.g., AS, VS, and depolarization signals associated with fibrillation which are sometimes referred to as “F-waves” or “Fib-waves”) can be classified by the microcontroller 160 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks). The arrhythmia detector 162, mentioned above, can be used to detect and characterize such arrhythmias.

Cardiac signals are also applied to the inputs of an analog-to-digital (A/D) data acquisition system 190. The data acquisition system 190 is configured to acquire intracardiac electrogram signals, convert the raw analog data into a digital signal, and store the digital signals for later processing and/or telemetric transmission to an external programmer 104 or a bedside monitor or personal advisory module (PAM) 102. The data acquisition system 190 is coupled to the RA lead 120, the LV lead 124, and the RV lead 130 through the switch 174 to sample cardiac signals across any pair of desired electrodes. The microcontroller 160 is further coupled to a memory 194 by a suitable data/address bus 196, wherein the programmable operating parameters used by the microcontroller 160 are stored and modified, as required, in order to customize the operation of pacemaker/ICD 100 to suit the needs of a particular patient. Such operating parameters define, for example, the amplitude or magnitude, pulse duration, electrode polarity, for both pacing pulses and impedance detection pulses as well as pacing rate, sensitivity, arrhythmia detection criteria, and the amplitude, waveshape and vector of each pacing and shocking pulse to be delivered to the patient's heart within each respective tier of therapy. Other pacing parameters include base rate, rest rate and circadian base rate.

Advantageously, the operating parameters of the implantable pacemaker/ICD 100 may be non-invasively programmed into the memory 194 through a telemetry circuit 101 in telemetric communication with an external device 104 or bedside monitor 102, such as a programmer, transtelephonic transceiver or a diagnostic system analyzer. The telemetry circuit 101 is activated by the microcontroller by a control signal 106. The telemetry circuit 101 advantageously allows intracardiac electrograms and status information relating to the operation of pacemaker/ICD 100 (as contained in the microcontroller 160 or memory 194) to be sent to the external device 102 through an established communication link 103. An internal warning device 121 (also referred to as a patient alert) may be provided for generating perceptible warning signals to the patient via vibration, voltage or other methods.

Pacemaker/ICD 100 further includes an accelerometer or other physiologic sensor 108, commonly referred to as a “rate-responsive” sensor because it is typically used to adjust pacing stimulation rate according to the exercise state of the patient. However, the physiological sensor 108 may further be used to detect changes in cardiac output, changes in the physiological condition of the heart, or diurnal changes in activity (e.g., detecting sleep and wake states) and to detect arousal from sleep. Accordingly, the microcontroller 160 can respond by adjusting the various pacing parameters (such as rate, AV delay, VV delay, etc.) at which the atrial and ventricular pulse generators, 170 and 172, generate stimulation pulses. While shown as being included within pacemaker/ICD 100, it is to be understood that the physiologic sensor 108 may also be external to pacemaker/ICD 100, yet still be implanted within or carried by the patient. A common type of rate responsive sensor is an activity sensor incorporating an accelerometer or a piezoelectric crystal, which is mounted within the housing 140 of pacemaker/ICD 100. Other types of physiologic sensors are also known, for example, sensors that sense the oxygen content of blood, respiration rate and/or minute ventilation, pH of blood, ventricular gradient, stroke volume, cardiac output, contractility, etc.

The pacemaker/ICD additionally includes a battery 110, which provides operating power to all of the circuits shown in FIG. 13. The battery 110 may vary depending on the capabilities of pacemaker/ICD 100. If the system only provides low voltage therapy, a lithium iodine or lithium copper fluoride cell typically may be utilized. For pacemaker/ICD 100, which employs shocking therapy, the battery 110 should be capable of operating at low current drains for long periods, and then be capable of providing high-current pulses (for capacitor charging) when the patient requires a shock pulse. The battery 110 should also have a predictable discharge characteristic so that elective replacement time can be detected. Accordingly, appropriate batteries are employed.

As further shown in FIG. 1B, pacemaker/ICD 100 is shown as having an impedance measuring circuit 112, which is enabled by the microcontroller 160 via a control signal 114. Uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring respiration; and detecting the opening of heart valves, etc. The impedance measuring circuit 112 is advantageously coupled to the switch 174 so that any desired electrode may be used.

In the case where pacemaker/ICD 100 is intended to operate as an implantable cardioverter/defibrillator (ICD) device, it detects the occurrence of an arrhythmia, and automatically applies an appropriate electrical shock therapy to the heart aimed at terminating the detected arrhythmia. To this end, the microcontroller 160 further controls a shocking circuit 173 by way of a control signal 179. The shocking circuit 173 generates shocking pulses of low (up to 0.1 joules), moderate (0.1-10 joules) or high energy (11 to 40 joules or more), as controlled by the microcontroller 160. Such shocking pulses are applied to the heart of the patient through at least two shocking electrodes, and as shown in this embodiment, selected from the LA coil electrode 128, the RV coil electrode 136, and/or the SVC coil electrode 138. The housing 140 may act as an active electrode in combination with the RV electrode 136, or as part of a split electrical vector using the SVC coil electrode 138 or the LA coil electrode 128 (i.e., using the RV electrode as a common electrode). Cardioversion shocks are generally considered to be of low to moderate energy level (so as to minimize pain felt by the patient), and/or synchronized with a R-wave and/or pertaining to the treatment of tachycardia. Defibrillation shocks are generally of moderate to high energy level (i.e., corresponding to thresholds in the range of 7-40 joules), delivered asynchronously (since R-waves may be too disorganized), and pertaining exclusively to the treatment of fibrillation. Accordingly, the microcontroller 160 is capable of controlling the synchronous or asynchronous delivery of the shocking pulses.

The above described implantable device 100 was described as an exemplary pacemaker/ICD. One or ordinary skill in the art would understand that embodiments of the present invention can be used with alternative types of implantable devices. Accordingly, embodiments of the present invention should not be limited to use only with the above described device.

### Pre-Pacing Pulses

Embodiments of the present invention, which are described below, generally involve delivering pre-pacing pulses to one or more sites within the LV chamber, and analyzing evoked responses to the pre-pacing pulses.

Pre-pacing pulses, as the term is used herein, are pacing pulses delivered prior to intrinsic activations of the LV chamber for the purpose of detecting and analyzing evoked responses to the pre-pacing pulses. Pre pacing pulses need not be delivered for a therapeutic purpose, e.g., they need not be delivered to attempt to optimize AV delay, synchrony and/or filling times. Nevertheless, pre-pacing pulses may have a therapeutic effect. Further, it is noted that where a patient's LV chamber is already being pacing using a specified AV delay and/or RV-LV delay, therapeutic pacing pulses delivered to the LV chamber can function as the pre-pacing pulses so long as they are delivered early enough to avoid fusion with an intrinsic LV activation.

One way to determine appropriate timing for delivering pre-pacing pulses involves pacing the patient's atrium for a plurality of beats (to normalize heart rate), and determining the average AV delay between the paced atrial activations and intrinsic ventricular activations. Once the average AV delay is determined, a time for delivering pre-pacing pulses following paced (or intrinsic) atrial activations can be equal to the average AV delay minus a safety margin (e.g., 10 ms). The safety margin is preferably long enough to avoid fusion with an intrinsic activation of the LV chamber. Another way to determine appropriate timing for delivering pre-pacing pulses involves measuring the delay between intrinsic atrial activations and intrinsic ventricular activations for a plurality of intrinsic heart beats, and then averaging the measured delays. A time for delivering pre-pacing pulses following paced (or intrinsic) atrial activations can be equal to the determined average delay minus a safety margin (e.g., 10 ms). These are just a few examples, which are not meant to be limiting.

### Predict Imminent VT or VF

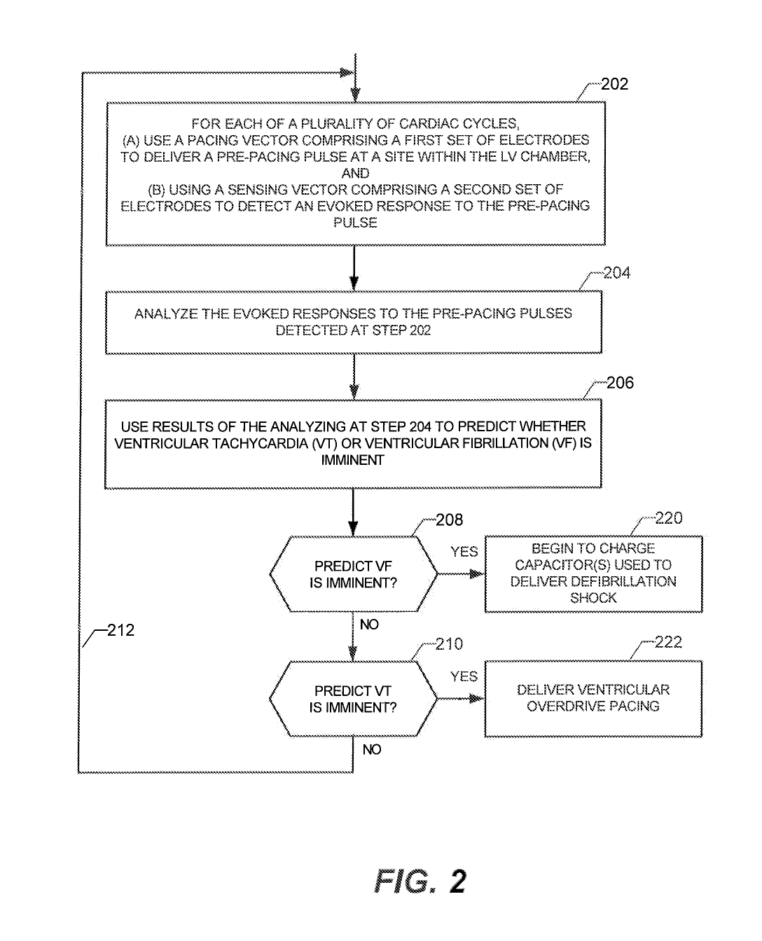

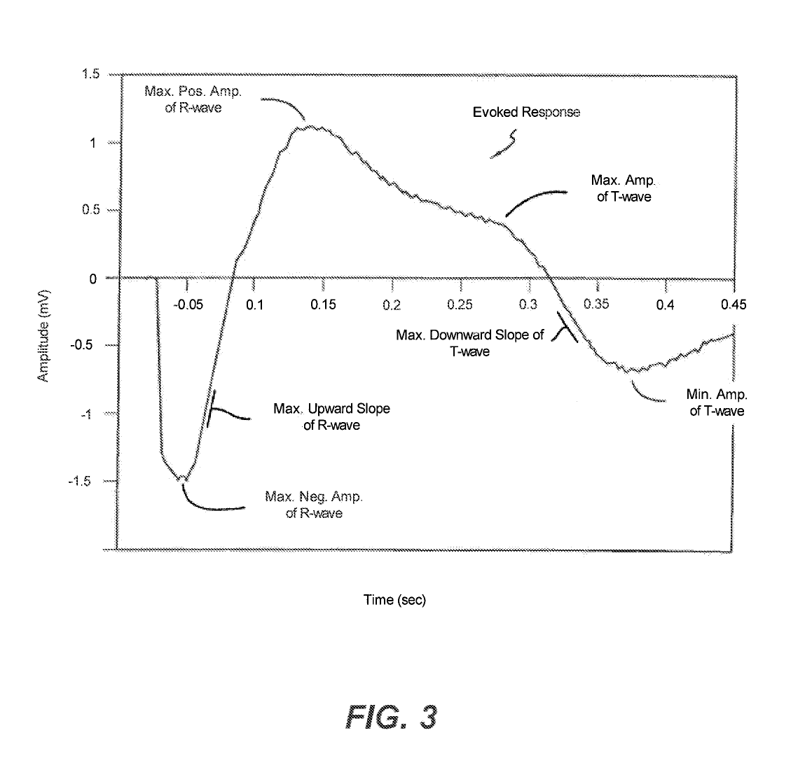

As mentioned above, it would be advantageous to predict the imminent onset of VT and VF, with the aim of either preventing VT and VF, preventing VT from progressing into VF, or shortening the duration of VT or VF. Certain embodiments of the present invention, which will be described with reference to FIGS. 2 and 3, can be used predict whether VT or VF is imminent, so that an appropriate response can be performed.

Reference shall first be made to FIG. 2, which is a high level flow diagram that is used to describe techniques to predict whether VT or VF is imminent, according to embodiments of the present invention.

Referring to FIG. 2, at step 202, for each of a plurality of cardiac cycles (e.g., 10 cardiac cycles, but not limited thereto), a pacing vector is used to deliver a pre-pacing pulse at a site within the LV chamber, and a sensing vector is used to detect an evoked response to the pre-pacing pulse. The pacing vector includes a first set of electrodes, with at least one electrode configured as an anode and at least one electrode configured as a cathode. In accordance with an embodiment, at least the cathode electrode(s) of the pacing vector is/are located within the LV chamber. The sensing vector includes a second set of electrodes, with at least one electrode configured as an anode and at least one electrode configured as a cathode. In accordance with an embodiment, at least the cathode electrode(s) of the sensing vector is/are located within the LV chamber. In one embodiment, the first set of electrodes (used to provide the pacing vector) and second set of electrodes (used to provide the sensing vector) include the same electrodes. However, to better detect evoked responses to the pre-pacing pulses, it is preferable that at least the cathode electrode(s) used to provide the sensing vector is/are a distance from the cathode electrode(s) used to provide that pacing vector. For example, referring briefly back to FIG. 1A, an exemplary pacing vector can include the D1 electrode 126₁ connected as the cathode and the RV coil 136 connected as the anode; and an exemplary sensing vector can include the P4 electrode 126₄ connected as the cathode and the RV coil 136 connected as the anode. This is just one example, which is not meant to be limiting.

At step 204, the evoked responses to the pre-pacing pulses (detected at step 202) are analyzed. At step 206, results of the analyzing (at step 204) are used to predict whether VT or VF is imminent. Additional details of steps 204 and 206 are explained below.

In accordance with certain embodiments, step 204 includes determining one or more predetermined features of each of the evoked responses to the pre-pacing pulses detected at 202, and determining a measure of variation for each of the one or more predetermined features. In such embodiments, step 206 can include using the one or more measures of variation determined at step 204 to predict whether VT or VF is imminent. More specifically, it is believed that such measures of variation of evoked responses will increase when VT is imminent, and increase even more so when VF is imminent.

In accordance with an embodiment, the one or more predetermined features of each of the evoked responses includes one or more features of an R-wave (indicative of ventricular depolarization) and/or one or more features of a T-wave (indicative of ventricular repolarization). FIG. 3 illustrates an exemplary evoked response to a pre-pacing pulse. Specific exemplary features of an evoked response to a pre-pacing pulse, some of which are labeled in FIG. 3, include: the maximum upward slope of the R-wave, the maximum amplitude of the R-wave, the maximum downward slope of the R-wave, the maximum negative dV/dt of the R-wave, and peak-to-peak amplitude of a QRS complex. Another exemplary feature of an evoked response to a pre-pacing pulse is the time from delivery of the pre-pacing pulse to a predetermined feature (e.g., maximum amplitude) of the R-wave, many examples of which were just listed. Further exemplary features of an evoked response to a pre-pacing pulse include the number of deflections of the R-wave and the number of peaks of the R-wave. The integral of the R-wave (also known as paced depolarization integral (PDI) or area under the R-wave), is another exemplary, feature of an evoked response to a pre-pacing pulse. Further exemplary features of an evoked response include Fast Fourier Transform (FFT) features of the R-wave. Still further features include a maximum amplitude of the T-wave, the integral of the T-wave (also known as area under the T-wave), time from delivery of the pre-pacing pulse to onset of the T-wave, and time from delivery of the pre-pacing pulse to peak of the T-wave.

The measure of variation can be a measure of: standard deviation, normalized standard deviation, interquartile range, range, mean difference, median absolute deviation, average absolute deviation, coefficient of variation, quartile coefficient of dispersion, relative mean difference, variance, or variance-to-mean ratio of the metric, but is not limited thereto. Another potential measure of variation that can be determined is a determination of whether or not there exists a bimodal distribution, which is indicative of alternans. More generally, it is also within the scope of the present invention to analyze the evoked responses to the pre-pacing pulses at step 204 by determining an extent of beat-to-beat alternans associated with the evoked responses. In such an embodiment, step 206 can include predicting whether VT or VF is imminent based on the extent of beat-to-beat alternans determined at step 204. In other words, the extent of beat-to-beat alternans can be a measure of variation.

Preferably, the one or more features of evoked responses determined at step 204 (as part of the analysis of the evoked responses to pre-pacing pulse) is/are of the same type(s), so that such feature(s) can be readily compared. For example, each time step 204 is performed, the maximum amplitude of the R-wave and the area under the R-wave, can be determine for 10 cardiac cycles. A measure of variation (e.g., standard deviation) can then be determined for the maximum amplitude of the R-wave for the 10 cardiac cycles, and a measure of variation (e.g., standard deviation) can be determined for the area under the R-wave for the 10 cardiac cycles. The two measures of variation can then be combined, e.g., using a weighted average, which may or may not be equally weighted. Alternatively, the two (or more) measures of variation need not be combined. It is also possible that only a single measure of variation is determined for a single predetermined feature of evoked responses to prem pacing pulses. These are just examples, which are not meant to be limiting.

Referring again to FIG. 2, step 206 can include comparing the one or more measures of variation to one or more VT variation thresholds that if exceeded is/are indicative of VT being imminent. Step 206 can also include comparing the one or more measures of variation to one or more VF variation thresholds that if exceeded is/are indicative of VF being imminent. Each VF variation threshold is greater than a corresponding VT variation threshold. The prediction of whether VT or VF is imminent can be based on results of such comparisons.

For example, where there is only one measure of variation to compare to the VT and VF variations thresholds, imminent VF can be predicted if the VF variation threshold is exceeded, and imminent VT can be predicted if the VT variation threshold (but not the VF variation threshold) is exceeded. If there are multiple measures of variation to compare to multiple VF variation thresholds and multiple VT variation thresholds, then imminent VF can be predicted if at least one VF variation threshold is exceeded, and imminent VT can be predicted if at least one VT variation threshold (but not the corresponding VF variation threshold) is exceeded. Alternatively, where there are multiple measures of variation (e.g., N measures) to compare to multiple VF variation thresholds and multiple VT variation thresholds, it can be that imminent VF is only predicted if all of the VF variation thresholds are exceeded, and imminent VT is only predicted if all of the VT variation thresholds (but not all of the corresponding VF variation thresholds) are exceeded. In still another embodiment, where there are multiple measures of variation (e.g., N measures) to compare to multiple VF variation thresholds and multiple VT variation thresholds, it can be that imminent VF is predicted if at least M out of N of the VF variation thresholds are exceeded, and imminent VT is predicted if at least M out of N of the VT variation thresholds (but not M out of N of the corresponding VF variation thresholds) are exceeded (where M and N are integers, and M<N). Other variations are also possible.

As indicated by steps 208 and 220, one or more capacitors used for delivering a defibrillation shock can begin to be charged, in response to predicting that VF is imminent. Such charging of the capacitor(s) enables a shock to be delivered more quickly if and when VF is detected. Additionally, or alternatively, in response to predicting that VF is imminent, overdrive pacing can be delivered to attempt to prevent VF from occurring. Additionally, in response to predicting that VF is imminent, an alert can be triggered to notify medical personal of the imminent VF in case the patient requires medical attention after VF occurs.

As indicated at steps 210 and 222, ventricular overdrive pacing can be delivered in response to predicting that VT is imminent. Such ventricular overdrive pacing is delivered to reduce a chance of VT sustaining or accelerating into a fast polymorphic VT or VF. Additionally, or alternatively, one or more sensors and/or algorithms that measure hemodynamic stability can be enabled in response to predicting that VT is imminent. Such sensors and/or algorithms could be used discriminate between a VT that can be treated with ATP or left to self-terminate, versus one which should be treated more aggressively by a shock.

As indicated by line 212, where imminent VT or VF is not predicted (or, is no longer predicted), steps 202-210 can be repeated. For example, these steps can be continually repeated, repeated every minute, repeated every hour, or repeated at some other time interval. It is also possible that there is a triggering event that causes these steps to be performed.

One of the embodiments of the present invention described herein can be used as the sole technique for predicting imminent VT or VF. Alternatively, a plurality of the embodiments described herein can be used (e.g., in parallel) for predicting imminent VT or VF. It is also possible that one or more embodiments described herein can be used in combination with other techniques for predicting imminent VT or VF.

### Estimate Ischemic Burden

As mentioned above, a higher ischemic burden has been shown to be correlated with a greater chance of future adverse coronary events and greater morbidity and mortality in patients. Accordingly, it would be beneficial to be able to chronically estimate ischemic burden and detect changes in such estimates. Also, as explained below, an alert can be triggered based on an estimate of ischemic burden. Alternatively, or additionally, CRT therapy can be triggered and/or adjusted based on an estimate of ischemic burden.

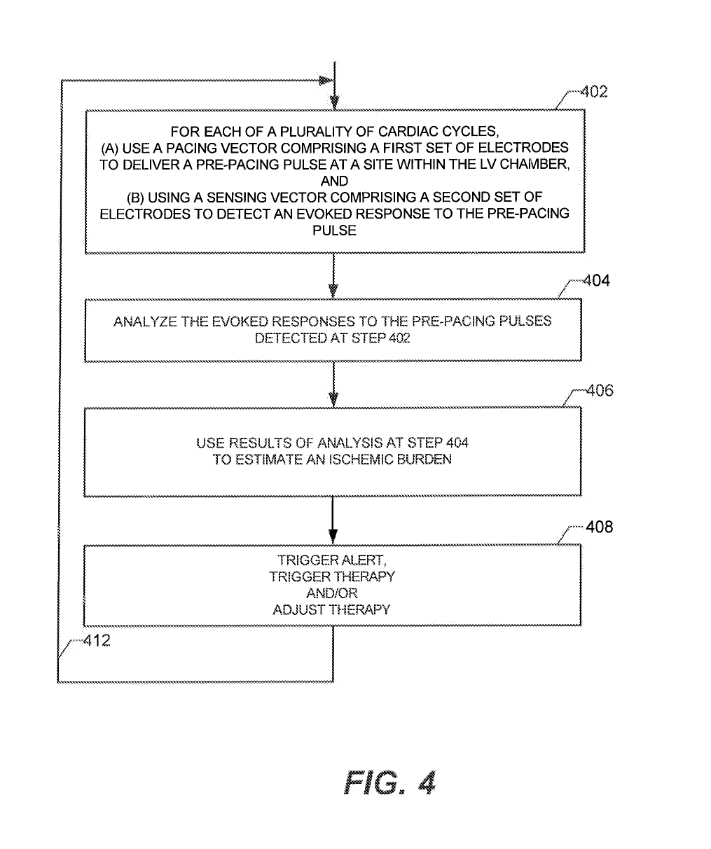

Reference is now made to FIG. 4, which is a high level flow diagram that is used to describe techniques to estimate a patient's ischemic burden. Referring to FIG. 4, at step 402, for each of a plurality of cardiac cycles (e.g., 10 cardiac cycles, but not limited thereto), a pacing vector is used to deliver a pre-pacing pulse at a site within the LV chamber, and a sensing vector is used to detect an evoked response to the pre-pacing pulse. At step 404, the evoked responses to the pre-pacing pulses (detected at step 402) are analyzed. At step 406, results of the analyzing (at step 404) are used to estimate ischemic burden.

Step 402 is substantially the same as step 202 discussed above with reference to FIG. 2. Accordingly, additional details of step 402 can be appreciated from the above discussion of step 202.

In accordance with certain embodiments, step 404 includes determining one or more predetermined features of each of the evoked responses to the pre-pacing pulses detected at step 402, and determining a measure of variation for each of the one or more predetermined features. Additional details regarding determining features of evoked responses to the pre-pacing pulses, and determining measures of variation of such features, can be appreciated from the above discussion of step 204 in FIG. 2.

Another measure of variation that can be determined is a determination of whether or not there exists a bimodal distribution, which is indicative of alternans. More generally, it is also within the scope of the present invention to analyze the evoked responses to the pre-pacing pulses by determining an extent of beat-to-beat alternans associated with the evoked responses. In such an embodiment, step 406 can include estimating ischemic burden based on the extent of beat-to-beat alternans determined at step 404. In other words, the extent of beat-to-beat alternans can be a measure of variation.

It is believed that the greater the measure(s) of variation (e.g., the greater the extent of beat-to-beat alternans), the greater the ischemic burden. Accordingly, increases in the measure(s) of variation over time are indicative of increases in ischemic burden. Conversely, decreases in measure(s) of variation over time are indicative of decreases in ischemic burden. Where the measure(s) of variation remain substantially constant over time, the ischemic burden has remained substantially the same. Additionally, the degree of complex fractionation (e.g., via FFT analysis) of the evoked responses to pre-pacing pulses can be used as a factor in quantifying ischemic burden, where a greater degree of fractionation would correspond to a greater ischemic burden. Also, the presence of double potentials could factor into a higher score for total ischemic burden.

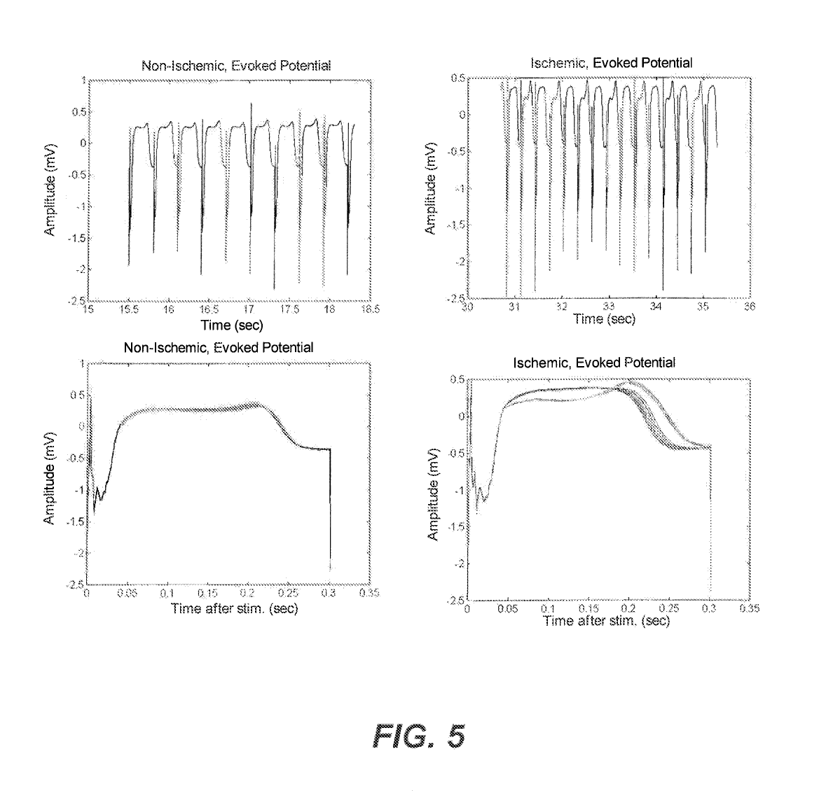

FIG. 5 illustrates morphologies of evoked responses to pre-pacing pulses where a subject has no myocardial ischemia, as well as where a subject has myocardial ischemia. The upper left graph illustrates baseline evoked responses for a heart (a rabbit, Langendorf preparation) without ischemia. In the lower left graph, the evoked responses of the upper left graph are aligned one above the other, illustrating that there are no morphology alternans, and more generally, that there is substantially no morphology variation. The upper right graph illustrates evoked responses for a heart (a rabbit, Langendorf preparation) with ischemia. In the lower right graph, the evoked responses of the upper right graph are aligned one above the other, illustrating that there are morphology alternans, and more generally, that there is a substantial morphology variation.

There are numerous ways to determine an extent of evoked response alternans. For example, a feature (e.g., maximum amplitude) of even numbered evoked responses can be averaged, and the same feature (e.g., maximum amplitude) of odd numbered evoked responses can be averaged, and the two averages can be compared to one another. The further apart the two averages are from one another, the greater the extent of alternans, and thus the greater the ischemic burden. The closer the two averages are to one another, the lower the extent of alternans, and thus the lower the ischemic burden.

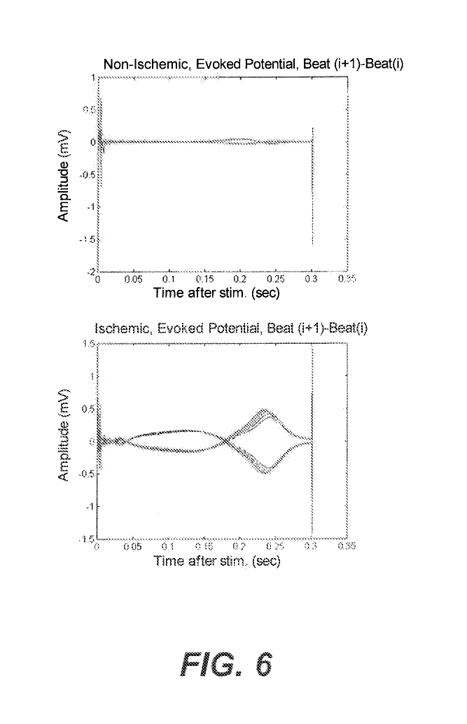

For another example, the even evoked response morphologies can be averaged (to produced an averaged even evoked response morphology), and the odd evoked response morphologies can be averaged (to produce an averaged odd evoked response morphology), and the averaged even evoked response morphology can be compared to the averaged odd evoked response morphology. The greater the distinction between the averaged even evoked response morphology and the averaged odd evoked response morphology, the greater the extent of alternans, and thus the greater the ischemic burden. The more similar the averaged even evoked response morphology and the averaged odd evoked response morphology are to one another, the lower the extent of alternans, and thus the lower the ischemic burden. In a specific example, an estimate of ischemic burden can be quantified by a summation of the absolute value difference on a beat-to-beat basis of evoked responses. Referring to FIG. 6, the upper graph illustrates beat-to-beat morphology differences for evoked responses to pre-pacing pulses where a subject has no myocardial ischemia. The lower graph in FIG. 6 illustrates beat-to-beat morphology differences where a subject has myocardial ischemia.

As another example of a way to determine the extent of evoked response alternans, a fast Fourier transform (FFT) of the evoked response signal can be taken, and if there is a large peak in signal power at half the pacing frequency, then alternans is determined to be present. Similarly, if there is a large peak at 3× or 4× the pacing frequency that could also be considered alternans, as ABCABC or ABCDABCD patterns, respectively (that is, alternans need not always be 1:1 or ABAB type).

In accordance with other embodiments of the present invention, the analysis performed at step 404 can include determining a conduction time delay from each pre-pacing pulse to a predetermined feature of an R-wave or T-wave of an evoked response to the pre-pacing pulse, and step 406 can include estimating an ischemic burden based on lengths of the conduction time delays. Examples of predetermined features of an R-wave or T-wave of an evoked response to a pre-pacing pulse (which can be determined at step 404) were discussed above with reference to step 204 of FIG. 2 and with reference to FIG. 3, and thus, need not be repeated. For a specific example, where the plurality of cardiac cycles referred to in step 402 equals ten, and the predetermined feature is maximum amplitude of an R-wave, step 404 can include determining a conduction time delay from each pre-pacing pulse (of the ten pre-pacing pulses) to a maximum amplitude of an R-wave immediately following the pre-pacing pulse. Step 406 can then include averaging the ten conduction time delays, and estimating an ischemic burden based on the average conduction time delay. For such embodiments, it is believed that the greater the conduction time delay (e.g., compared to a baseline conduction time delay) the higher the ischemic burden. Alternatively, or additionally, step 406 can include determining a variation of the conduction time delays, and estimating an ischemic burden based on the variation of conduction time delays. For such embodiments, it is believed that the greater the variation the higher the ischemic burden.

As mentioned above, at step 406, results of the analyzing (at step 404) are used to estimate ischemic burden. Step 406 can include comparing the one or more measures of variation and/or conduction time delay, determined at step 404, to one or more corresponding thresholds, to estimate the patient's ischemic burden. For example, a single threshold can be used to classify the ischemic burden as either high or low. Two thresholds can be used to classify the ischemic burden as low, medium or high. It is also possible that additional thresholds be used to provide additional levels of granularity. Where two or more features of each of the evoked responses to the pre-pacing pulses are determined, and a measure of variation is determined for each feature, there can be one or more ischemic burden thresholds for which to compare to each measure of variation. It is also possible that one or more measures of variation and/or conduction time delay be plugged into an algorithm that is used to calculate a value or other metric indicative of estimated ischemic burden. Such thresholds can be defined based on data collected from a broad patient population and/or tailored for specific patients. Other variations are also possible.

As indicated at step 408, the estimate of ischemic burden (determined at step 406) can be used to selectively trigger an alert, trigger therapy and/or adjust therapy. For example, where the estimate of ischemic burden (determined at step 406), or the measure of variation and/or conduction time (determine at step 406), exceeds a corresponding threshold(s), a patient alert (e.g., using warning device 121) can be triggered. Alternatively, or additionally, CRT pacing can be triggered, or the number of pacing sites used for delivering CRT pacing can be adjusted. For a more specific example, if the patient is currently being paced using single site LV pacing, and the estimated ischemic burden crosses a certain programmable threshold (e.g., “ischemic threshold A”), then dual-site may pacing (BiV pacing at two LV sites) can be initiated. If the estimated ischemic burden crosses a higher programmable threshold (e.g., “ischemic threshold B”), then a more aggressive triple-site MSLV pacing (BiV pacing at three LV sites) can be initiated.

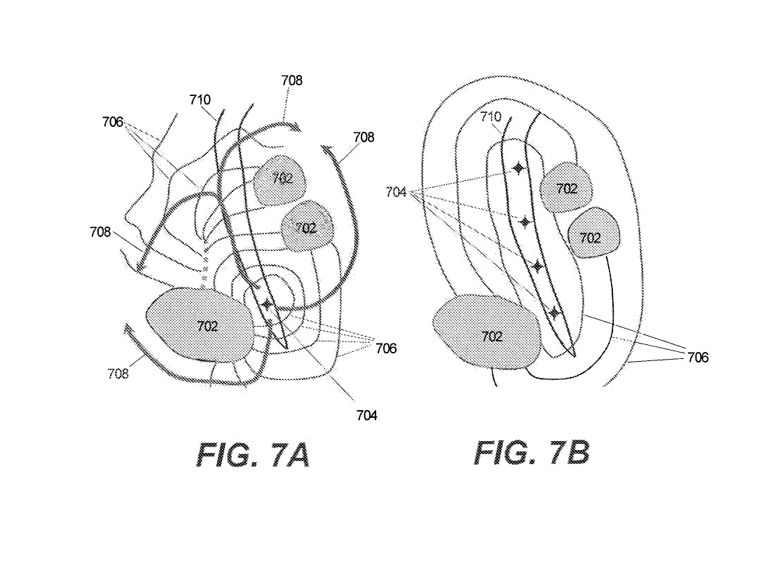

Benefits of increasing the number of LV pacing sites can be appreciated from a comparison of FIGS. 7A and 7B. In these FIGS., the gray zones 702 represent zones of ischemia (also referred to as ischemic zones) within an LV chamber. These zones of ischemia 702 can create lines of structural and/or functional block, which slow down electrical conduction, leading to sub-optimal dyssynchronous mechanical activation and reduced pump efficiency of the heart. In FIG. 7A, the single diamond 704 represents a single LV pacing site, and the lines 706 represent wavefront propagations from the single LV pacing site 704. The thicker arrowed lines 708 represent paths of depolarization wave propagation. In FIGS. 7A and 7B line 710 generally represents a border tracing of the LV pacing lead. In FIG. 7B, the multiple diamonds 704 represent multiple LV pacing sites (four pacing sites 704, in this example). Notice that in FIG. 7B, the depolarizations induced at the multiple LV pacing sites 704 produce relatively flat wavefront propagations 706 throughout the LV tissue that may have sufficient energy to break through lines of functional block created by the ischemic zones 702.

It is also noted that MSLV pacing lowers the chance of tachyarrhythmias from re-entrant circuits, triggered by impulses (intrinsic or suboptimal single-site LV pacing) near the ischemic sites. In accordance with specific embodiments, the timing between LV pulses can also be adjusted to attempt to prevent induction of reentrant circuits.

In the above described embodiments, the pre-pacing pulses can be delivered at a single pacing site within the LV chamber. It is also possible that pre-pacing pulses can be delivered at multiple sites within the LV chamber, simultaneously, or with a delay therebetween.

In certain embodiments, pre-pacing pulses are delivered at a single site for a plurality of cardiac cycles, and at two sites for a further plurality of cardiac cycles. In such embodiments, as part of the analysis of the evoked responses, evoked responses to the two site pre-pacing pulses can be compared to the evoked responses to the single site pre-pacing pulses to estimate ischemic burden and/or characterize an electrical substrate of the LV chamber. This can include using results of the comparison to estimate a location of an ischemic region within the LV chamber.

For a specific example, pre-pacing pulses can be delivered for a plurality of cardiac cycles (e.g., 10 cardiac cycles) using the M2 electrode 126₂ (in FIG. 1A) within the LV chamber configured as the cathode and the case electrode 140 (in FIG. 1B) or the RV coil 136 (in FIG. 1A) configured as the anode. In other words, such electrodes can be the set of electrodes making up a pacing vector. One or more sensing vectors can be used to sense the evoked responses to the pre-pacing pulses delivered to the single site within the LV. For example, a first sensing vector can include the P4 electrode 126₄ within the LV chamber and the case electrode 140 (or the P4 electrode 126₄ and the M3 electrode 126₃), and a second sensing vector can include the RV tip electrode 132 and RV ring electrode 134. As part of the analysis of the evoked responses, there can be a determination of the conduction time delays from each pre-pacing pulse to a predetermined feature of an R-wave or T-wave of an evoked response to the pre-pacing pulse. For a further plurality of cardiac cycles (e.g., a further 10 cardiac cycles), pre-pacing pulses can be delivered at two sites within the LV chamber, e.g., one site being proximate the M2 electrode 126₂ and a second site being proximate that D1 electrode 126₁ (i.e., M2 and D1 can both be configured as a cathode). The same two sensing vectors can be used to sense the evoked responses to the pre-pacing pulses delivered to the two sites within the LV, and conduction time delays can be determined. If the conduction time delays to the two sensing sites (e.g., sites proximate the P4 electrode and the RV tip electrode) in response to the two site pre-pacing pulses are faster than the conduction time delays in response to the single site pre-pacing pulses, this can be interpreted as being indicative of no or a very low ischemic burden within the LV chamber. If instead conduction time delays to the P4 electrode sensing site were unchanged with dual-site pre pacing pulse pacing while the conduction time delays to the RV tip electrode sensing site are faster (i.e., shorter) with dual-site pre-pacing pulse pacing, this can be interpreted as there being a change in the LV substrate indicating ischemia somewhere “between” (in an activation propagation sense) the pacing electrodes and the P4 sense electrode. Finally, if instead the paced conduction times to both sensing sites are unchanged whether performing single site pre-pacing pulse pacing or dual-site pre-pacing pulse pacing within the LV chamber, this can be interpreted as there being an ischemic substrate at or near the second pacing site (in this example, the site proximate the D1 electrode) resulting in slow conduction and/or exit block.

Faster conduction time delays in response pre-pacing pulses delivered to two sites within the LV chamber, as compared to in response to pre-pacing pulses delivered at a single site within the LV chamber, can occur in at least two scenarios: exit block and/or slow conduction (due to functional or ischemic cause) at or near the sensing site; or alteration of the activation wavefront path and/or conduction velocity resulting from pacing at the second site. Accordingly, other interpretations (than those described above) of changes in conduction time delays are also possible.

Referring again to FIG. 4, as indicated by line 412, steps 402-408 can be repeated. For example, these steps can be continually repeated, repeated every minute, repeated every hour, or repeated at some other time interval. It is also possible that there is a triggering event that causes these steps to be performed.

One of the embodiments of the present invention described herein can be used as the sole technique for estimating ischemic burden. Alternatively, a plurality of the embodiments described herein can be used (e.g., in parallel) for estimating ischemic burden. It is also possible that one or more embodiments described herein can be used in combination with other techniques for estimating ischemic burden.

### Characterize Electrical Substrate of LV Chamber

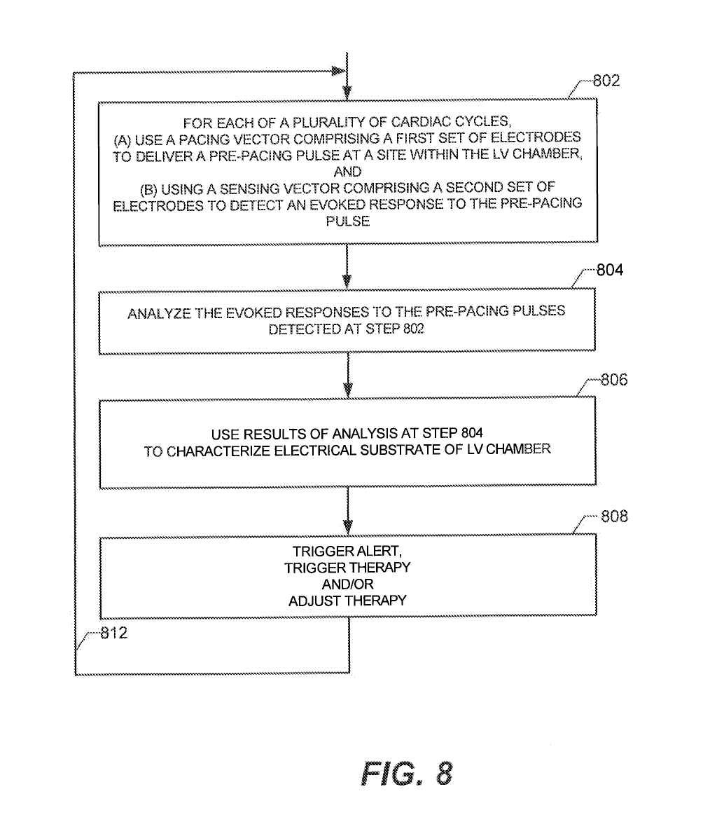

The flow diagram of FIG. 4 was used to describe how embodiments of the present invention can be used to estimate a patient's ischemic burden. The same or similar concepts can be used to more generally characterize an electrical substrate of the LV chamber, as shown in the flow diagram of FIG. 8.

Steps 802 and 804 are substantially the same as steps 402 and 404, and thus, need not be explained again. Step 806 involves using results of the analysis at step 804 to characterize an electrical substrate of the LV chamber, which can include, e.g., characterizing an extent of functional and/or structural block within the LV chamber and/or identifying a likely location of an ischemic substrate. For example, high measures of variation and/or high conduction time delays (or increases in the same) can be indicative of high functional and/or structural block within the LV chamber (or increases in functional and/or structural block). Various thresholds can be used to quantify the levels of functional and/or structural block, in a similar manner as was described above with reference to step 406. At step 808, the characterization of the electrical substrate of the LV chamber (determined at step 806) can be used to selectively trigger an alert, trigger therapy and or adjust therapy. For example, where the measure of variation and/or conduction time delay (determine at step 806), exceeds a corresponding threshold(s), a patient alert (e.g., using warning device 121) can be triggered. Alternatively, or additionally, CRT pacing can be triggered, or the number of pacing sites used for delivering CRT pacing can be adjusted.

Additionally, in manners similar to those described above, evoked responses to pre-pacing pulses delivered at a single site can be compared to evoked response to pre-pacing pulses delivered at multiple sites to estimate locations of functional and/or structural block (e.g., due to ischemic zones, but not limited thereto). Multiple sensing vectors can be used to more accurately estimate such locations.

As indicated by line 812, steps 802-808 can be repeated. For example, these steps can be continually repeated, repeated every minute, repeated every hour, or repeated at some other time interval. It is also possible that there is a triggering event that causes these steps to be performed.

One of the embodiments of the present invention described herein can be used as the sole technique for characterizing an electrical substrate of the LV chamber. Alternatively, a plurality of the embodiments described herein can be used (e.g., in parallel) for characterizing an electrical substrate. It is also possible that one or more embodiments described herein can be used in combination with other techniques for characterizing an electrical substrate of the LV chamber.

Embodiments of the present invention have been described above with the aid of functional building blocks illustrating the performance of specified functions and relationships thereof. The boundaries of these functional building blocks have often been arbitrarily defined herein for the convenience of the description. Alternate boundaries can be defined so long as the specified functions and relationships thereof are appropriately performed. Any such alternate boundaries are thus within the scope and spirit of the claimed invention. For example, it would be possible to combine or separate some of the steps shown in FIGS. 2, 4 and 8. For another example, it is possible to change the boundaries of some of the blocks shown in FIG. 1B.

The previous description of the preferred embodiments is provided to enable any person skilled in the art to make or use the embodiments of the present invention. While the invention has been particularly shown and described with reference to preferred embodiments thereof, it will be understood by those skilled in the art that various changes in form and details may be made therein without departing from the spirit and scope of the invention.

1. A method for use with an implantable system including a lead having one or more electrodes implantable in a patient's left ventricular (LV) chamber, the method comprising:
(a) for each of a plurality of cardiac cycles,
(a.1) using a pacing vector comprising a first set of electrodes to deliver a pre-pacing pulse at a site within the LV chamber, wherein the pre-pacing pulse is delivered prior to an intrinsic activation of the LV chamber; and
(a.2) using a sensing vector comprising a second set of electrodes to detect an evoked response to the pre-pacing pulse;
(b) analyzing the evoked responses to the pre-pacing pulses detected at step (a); and
(c) using results of the analyzing at step (b) to predict whether ventricular tachycardia (VT) or ventricular fibrillation (VF) is imminent, estimate ischemic burden and/or characterize an electrical substrate of the LV chamber.

2. The method of claim 1, wherein:
step (b) comprises analyzing the evoked responses to the pre-pacing pulses by
(b.1) determining one or more predetermined features of each of the evoked responses to the pre-pacing pulses detected at step (a); and
(b.2) determining a measure of variation for each of the one or more predetermined features; and
step (c) comprises using the one or more measures of variation determined at step (b) to predict whether VT or VF is imminent, estimate ischemic burden and/or characterize an electrical substrate of the LV chamber.

3. The method of claim 2, wherein the measure of variation for each of the one or more predetermined features is selected from the group consisting of:
standard deviation;
normalized standard deviation;
interquartile range;
range;
mean difference;
median absolute deviation;
average absolute deviation;
coefficient of variation;
quartile coefficient of dispersion;
relative mean difference;
variance; and
variance-to-mean ratio of the metric.

4. The method of claim 2, wherein step (c) comprises:
(c.1) comparing the one or more measures of variation to one or more VF variation thresholds that if exceeded is/are indicative of VF being imminent, wherein each VF variation threshold is greater than a corresponding VT variation threshold;
(c.2) comparing the one or more measures of variation to one or more VT variation thresholds that if exceeded is/are indicative of VT being imminent; and
(c.3) predicting whether VT or VF is imminent based on results of the comparing at steps (c.1) and (c.2).

5. The method of claim 2, wherein the one or more predetermined features of each of the evoked responses includes one or more features of an R-wave indicative of ventricular depolarization and/or one or more features of a T-wave indicative of ventricular repolarization.

6. The method of claim 2, wherein the one or more predetermined features of each of the evoked responses is/are selected from the group consisting of:
maximum upward slope of the R-wave;
maximum amplitude of the R-wave;
maximum downward slope of the R-wave;
maximum negative dV/dt of the R-wave;
time from delivery of the pre-pacing pulse to a predetermined feature of the R-wave;
number of deflections of the R-wave;
number of peaks of the R-wave:
integral of the R-wave;
a Fast Fourier Transform (FFT) feature;
maximum amplitude of the T-wave;
integral of the T-wave;
time from delivery of the pre-pacing pulse to onset of the T-wave; and
time from delivery of the pre-pacing pulse to peak of the T-wave.

7. The method of claim 2, wherein step (c) comprises:
(c.1) comparing each of the one or more measures of variation determined at step (b), or a combination thereof, to one or more corresponding thresholds: and
(c.2) predicting whether VT or VF is imminent based on results the comparing at step (c.1).

8. The method of claim 1, wherein step (c) comprises predicting whether VT or VF is imminent, and further comprising:
(e) in response to predicting that VF is imminent, beginning to charge one or more capacitors used for delivering a defibrillation shock, so that a shock can be delivered more quickly once VF is detected; and
d) in response to predicting that VT is imminent, delivering ventricular overdrive pacing to reduce a chance of VT sustaining or accelerating into a fast polymorphic VT or VF.

9. The method of claim 1, wherein:
step (b) comprises analyzing the evoked responses to the pre-pacing pulses by determining an extent of beat-to-beat alternans associated with the evoked responses; and
step (c) comprises predicting whether VT or VF is imminent, estimating an ischemic burden and/or characterizing an electrical substrate of the LV chamber based on the extent of beat-to-beat alternans determined at step (b).

10. The method of claim 1, wherein:
step (b) comprises analyzing the evoked responses to the pre-pacing pulses by determining a conduction time delay from each pre-pacing pulse to a predetermined feature of an R-wave or T-wave resulting from the pre-pacing pulse; and
step (c) comprises estimating an ischemic burden and/or characterizing an electrical substrate of the LV chamber based on the conduction time delays determined at step (h).

11. The method of claim 1, wherein:
step (b) comprises analyzing the evoked responses to the pre-pacing pulses by determining an extent of fractionation of portions of an electrocardiogram indicative of the evoked responses; and
step (c) comprises estimating an ischemic burden and/or characterizing an electrical substrate of the LV chamber based on the extent of fractionation determined at step (b).

12. The method of claim 1, wherein:
step (a.1) also includes using a further pacing vector comprising a further set of electrodes to deliver a further pre-pacing pulse at a further site within the LV chamber; and
step (a.2) includes using the sensing vector to detect an evoked response to the multiple pre-pacing pulses delivered at step (a.1).

13. The method of claim 1, wherein:
step (a.1) comprises using the pacing vector comprising the first set of electrodes to deliver a pre-pacing pulse at a single site within the LV chamber, wherein the first set of electrodes include only one cathode electrode within the LV chamber; and
step (a.2) comprises using the sensing vector comprising the second set of electrodes to detect an evoked response to the pre-pacing pulse delivered at the single site within the LV chamber;
step (a) also comprises, for each of a plurality of further cardiac cycles,
(a.3) using a pacing vector comprising a further set of electrodes, which includes two cathode electrodes within the LV chamber, to deliver a pre-pacing pulse at two sites within the LV chamber; and
(a.4) using the sensing vector comprising the second set of electrodes to detect an evoked response to the pre-pacing pulses delivered at the two sites within the LV chamber;
step (b) comprises analyzing the evoked responses to the pre-pacing pulses by
(b.1) determining, based on the evoked responses detected at step (a.2), conduction time delays that occur in response to the pre-pacing pulses delivered at the single site within the LV chamber at step (a.1);
(b.2) determining, based on the evoked responses detected at step (a.4), conduction time delays that occur in response to the pre-pacing pulses delivered at the two sites within the LV chamber at step (a.3); and
(b.3) comparing the conduction time delays determined at step (b.2) to the conduction time delays determined at step (b.1); and
step (c) comprises using results of the comparing at step (b.3) to estimate ischemic burden and/or characterize an electrical substrate of the LV chamber.

14. The method of claim 13, wherein:
step (c) comprises using results of the comparing at step (b.3) to estimate a location of an ischemic region within the LV chamber.

15. The method of claim 1, wherein step (c) comprises using results of the analyzing at step (b) to estimate ischemic burden; and further comprising:
(d) selecting how many sites within the LV chamber is/are to be paced, as part of cardiac resynchronization therapy (CRT), based on the ischemic burden estimated at step (c).

16. A method for use with an implantable system including a lead having one or more electrodes implantable in a patient's left ventricular (LV) chamber, the method comprising:
(a) delivering pre-pacing pulses to one or more sites within the LV chamber;
(b) detecting evoked responses the pre-pacing pulses;
(c) analyzing the evoked responses to the pre-pacing pulses; and
(d) using results of the analyzing to predict whether ventricular tachycardia (VT) or ventricular fibrillation (VF) is imminent, estimate ischemic burden and/or characterize an electrical substrate of the LV chamber.

17. An implantable system, comprising:
at least one lead having one or more electrodes implantable in a patient's left ventricular (LV) chamber;
one or more pulse generators configured to selectively generate pre-pacing pulses;
one or more sensing circuits configured to detect evoked response to pre-pacing pulses;
one or more processors configured to
analyze evoked responses to pre-pacing pulses: and
predict whether ventricular tachycardia (VT) or ventricular fibrillation (VF) is imminent, estimate ischemic burden and/or characterize an electrical substrate of the LV chamber based the analysis of the evoked responses to the pre-pacing pulses.

18. The implantable system of claim 17, wherein:
a pacing vector comprising a first set of electrodes delivers the pre-pacing pulses, selectively generated by the one or more pulse generators, at a site within the LV chamber, wherein each pre-pacing pulse is delivered prior to an intrinsic activation of the LV chamber; and
a sensing vector comprising a second set of electrodes is coupled to the one or more sensing circuits to detect evoked responses to the pre-pacing pulse.

19. The implantable system of claim 17, wherein the one or more processors is/are configured to:
determine one or more predetermined features of each of the evoked responses to the pre-pacing pulses;
determine a measure of variation for each of the one or more predetermined features; and
use the one or more measures of variation to predict whether VT or VF is imminent, estimate ischemic burden and/or characterize an electrical substrate of the LV chamber.

20. The implantable system of claim 17, wherein the one or more processor is/are configured to:
determine a conduction time delay from each pre-pacing pulse to a predetermined feature of an R-wave or T-wave resulting from the pre-pacing pulse; and
estimate ischemic burden and/or characterize an electrical substrate of the LV chamber based on the conduction time delays.
